(19) United States
(12) Patent Application Publication
(10) Pub. No.: US 2024/0368672 A1
(43) Pub. Date: Nov. 7, 2024

(54) BLOOD MATRIX AS REFERENCE MATERIAL FOR IN-VITRO DIAGNOSTICS

(71) Applicant: SENSID GMBH, Rostock (DE)

(72) Inventors: Björn NOWACK, Seebad-Warnemünde (DE); Angelika LÜKING, Ludwigsfelde (DE)

(21) Appl. No.: 18/580,717

(22) PCT Filed: Jul. 21, 2022
(86) PCT No.: PCT/EP2022/070560

§ 371 Date: Jan. 19, 2024

(30) Foreign Application Priority Data
Jul. 21, 2021 (EP) 21187066.2

(51) Int. Cl. C12Q 1/6806; C12Q 1/6844; C12Q 1/6869

(52) U.S. Cl. CPC C12Q 1/6806; C12Q 1/6846; C12Q 1/6869; G01N 2496/05

(57) ABSTRACT

The present invention relates both to a new reference material based on a blood matrix for use in diagnostics or in-vitro diagnostics, in particular those using sequencing methods of nucleic acids, such as polymerase chain reaction (PCR) or next generation sequencing (NGS), and to a method for its production including validation, in particular a non-invasive liquid biopsy (liquid biopsies).

The present invention relates both to a new reference material based on a blood matrix for use in diagnostics or in-vitro diagnostics, in particular those using sequencing methods of nucleic acids, such as polymerase chain reaction (PCR) or next generation sequencing (NGS), and to a method for its production including validation, in particular a non-invasive liquid biopsy (liquid biopsies).

Despite all the progress made in recent years, current methods for detecting tumours, such as clinical-chemical examinations of body fluids and tissue samples or imaging procedures, usually lead to malignant changes being detected too late. The main cause of the high mortality rate of tumour patients is therefore not the occurrence of primary tumours, but of metastases. In order to prevent the occurrence of metastases, malignant changes must be detected as early as possible. Furthermore, methods are needed to correctly distinguish malignant cells from normal cells. Both false-positive and false-negative findings have fatal consequences for those affected. Procedures are also required that allow tumour patients to be given a correct prognosis and allow treatment to be tailored to the individual patient.

So far, so-called tumour markers, in particular oncogenes, have been determined in body fluids such as blood, urine, sputum or tissue samples. These are components of tumour cells that are produced in increased or decreased amounts and whose changes can be detected in body fluids such as blood, plasma or serum.

The isolation, characterisation and analysis of intracellular components, especially nucleic acids (ribonucleic acids, RNA and deoxyribonucleic acids, DNA), is of great importance for modern molecular biology. Since the invention of the polymerase chain reaction (PCR) in 1983 at the latest, a large number of nucleic acid diagnostic methods have been developed which are used, for example, to detect diseases and pathogens.

In the state of the art, such cell material is prepared for diagnostics in the following steps, such as sample preparation, extraction, concentration, isolation, purification, reverse transcription if necessary, amplification and detection.

Analysing genetic cancer mutations is nowadays of central importance for patient management and therapy decisions. The assessment of the mutation profile in the context of cancer is usually carried out using genetic tests. The use of liquid biopsies from blood, which are based on the analysis of circulating tumour DNA (ctDNA) or cell free DNA (cfDNA), or RNA, e.g. that in exosomes or circulating tumour cells (CTC), has increased enormously in clinical practice in recent years, as it allows continuous, minimally invasive monitoring of the tumour genome over the course of the disease. With the proliferation of such tests specific to liquid biopsies and their use in routine diagnostics and increasing regulatory requirements, the need for validated assays performed to equivalent standards is also increasing.

The documentation of the clinical validity of a test also includes the evaluation of the analytical validity, which is described by the parameters accuracy, sensitivity, specificity and robustness of the test. The pre-analytical variables (or processes), which are factors without direct disease association but which affect the integrity of the body fluid or the biomarker (analyte) present in this body fluid or influence the results during the analysis and can be of technical, biological or environmental origin, have a major influence on these parameters. In the field of liquid biopsy tests, these are, for example, the extraction and quantification method used, storage conditions, PCR inhibition and fragment size bias. These pre-analytical variables can therefore lead to deviations between laboratories.

Due to the powerful amplification of tumour DNA using sequencing methods of nucleic acids, in particular PCR, “qPCR” (real-time quantitative polymerase chain reaction), “digital droplet PCR” (ddPCR), or “next generation sequencing” (NGS, or parallel sequencing such as “massive parallel sequencing”), so-called non-invasive liquid biopsy has been established in tumour diagnostics in recent years, whereby, for example, circulating free DNA in a sample is examined. Such circulating free DNA (cfDNA for short) can, for example, originate from a cancer/tumour cell (so-called ctDNA).

In cancer patients, the average cfDNA amount is higher than in healthy subjects, in particular the cfDNA plasma level is higher in advanced stage cancer patients than in mild/early stage cancer patients. For example, ctDNA accounts for 1% of cfDNA (0.01-90% of normal cfDNA, depending on the stage and location of the tumour).

However, there is a great need for a standard so that not only relative but also absolute diagnostic statements can be made. The use of such a standard is described, for example, in WO2018094183A1 or PCT/EP2021/050962 of the applicant.

Currently, both commercial tests and lab-developed tests (LDT) are available for mutations that need to be detected for tumour diagnostics, for example. Internal laboratory standards such as plasmid DNA or synthetic oligonucleotides are used as quality controls for validation and calibration.

A particular disadvantage is that these internal laboratory standards often do not adequately reflect the actual properties of the samples obtained from humans or animals, including the biomarkers or analytes they contain. Such a sample concerns the test material as it is obtained in a laboratory, e.g. blood, tissue, spinal fluid, mucosal swabs, sputum, urine, semen, saliva or other sample types.

Another disadvantage is that these internal laboratory standards have to be diluted for the purpose of validating or calibrating a device or procedure in in-vitro diagnostics and there is no automated process available for this, meaning that standardisation is usually prone to errors. Furthermore, the stability of such dilutions is not guaranteed and, depending on the source, e.g. plasmid DNA from E. coli, there is a risk of DNA/RNA contamination by this non-human or non-animal species, whereby even minimal entries are critical. Consequently, the copy number determined using PCR or NGS, for example, can be relatively and absolutely incorrect due to a lack of sufficient standardisation.

It is obvious that internal and external controls in the form of a reference material are required for the determination, evaluation and tracking of analytical performance, as well as for the quality monitoring of laboratories. The reference materials should have comparable physical and biological properties to the sample or starting material used. This in turn means that the focus is not only on the property of the biomarker, but also on the sample matrix in which, for example, a biomarker/analyte to be detected can be found. The target molecule of the liquid biopsy tests, e.g. ct/cfDNA (circular tumour/cell free) is found as cell-free DNA in the blood and is isolated from the blood after blood collection, subsequent plasma generation and DNA extraction. However, previous reference or control materials use DNA-free plasma or a DNA-free plasma-like solution as a basis, if necessary supplemented with a “biomarker/analyte of interest”, as blood as such, i.e. whole blood, usually has too many indeterminacies due to coagulation and haemolysis. Such indeterminacies can also result from sample handling (e.g. exposure to heat, contamination, completeness, etc.). As a consequence, however, the processes of sample handling (storage) and plasma generation or other preparation methods cannot be validated and evaluated. There is therefore a need to provide a blood sample as a reference material that largely corresponds to an original blood sample or simulates it sufficiently so that such a reference material can be compared with a patient blood (plasma, serum) sample. The patient blood sample may have been treated in any way.

In the case of a patient blood sample, storage or transport (temperature, duration) can cause the sample to be falsified due to coagulation, haemolysis and cell lysis and the resulting release of undesirable components, in particular genomic DNA, RNAses, DNAses and proteases. There is therefore a risk that such a sample may provide false-positive or false-negative analysis results, particularly with regard to the content or concentration of cfDNA or other nucleic acids.

It is also problematic that patient blood samples are extracted to obtain cfDNA or other nucleic acids, depending on personnel, equipment, etc. This can lead to low concentrations of cfDNA or other nucleic acids. A lack of extraction efficiency can lead to low concentrations of cfDNA or other nucleic acids, so that the required concentrations of cfDNA or other nucleic acids for performing a liquid biopsy are not achieved. On the other hand, there is an analytical need for the patient blood sample to be largely preserved, i.e. not limited to plasma or serum.

It is therefore important and necessary to improve standardisation in in-vitro diagnostics, especially those using nucleic acid sequencing methods.

Surprisingly, the inventors were able to establish that the use of a blood sample as a reference material in a diagnostic or in-vitro diagnostic, which may have a stabiliser, considerably improves the diagnostic quality. (a measurement is valid if it actually measures what it is supposed to measure and thus provides credible results),
reliability (reliability refers to whether a test system provides reliable results when performed repeatedly), and
objectivity (a test system is objective if there are no unwanted influences from persons involved) of a given test system. This is a particularly advantageous way of checking the performance parameters of the measurement. In particular, if a human or animal, especially from mammals, especially cattle, pigs or sheep, comparative blood sample is available.

The problem is solved by the technical teaching imparted by at least one patent claim.

The object of the present invention is therefore to provide an improved method for carrying out diagnostics or in-vitro diagnostics, wherein blood or in one of the following embodiments is used as a reference material to solve this problem.

Therefore, the invention relates to the use of i.) blood or ii.) blood cells and blood plasma or blood serum as reference material in a sample in a diagnostic or in-vitro diagnostic comprising a sequencing method of nucleic acids, wherein a stabiliser may be added. In a preferred embodiment of the invention, the stabiliser is to be selected in such a way that there is no influence on the biomarker or analyte to be determined.

Furthermore, the invention relates to the use of a reference material comprising blood in a sample in an in-vitro diagnostic comprising a sequencing method of nucleic acids, wherein blood plasma or blood serum is added to blood cells.

In addition, the invention relates to a method for the preparation of blood as reference material in a sample in in-vitro diagnostics comprising a sequencing method of nucleic acids, wherein blood plasma or blood serum is added to blood cells, and preferably one or more stabilisers are added.

Such stabilisers suitable according to the invention are preferably selected from the groups such as crosslinking stabilisers, alcoholic stabilisers and/or anticoagulants. Preferred are in particular formaldehyde, glutaraldehyde, glyoxal and acrolein, or mixtures thereof.

Furthermore, such stabilisers can be used, e.g. crosslinking stabilisers which produce covalent chemical bonds between proteins, such as, not exhaustively, selected from the group consisting of formaldehyde, paraformaldehyde, formalin, glutaraldehyde, osmium tetraoxide, imidoester crosslinkers, such as dimethyl suberimidate (DMS), acrolein, glyoxal, carbodiimide, or alcoholic stabilisers, such as, not conclusively, selected from the group ethanol, methanol, acetone, acetic acid, 2-phenoxyethanol, diethyl ether or anticoagulants/chelating agents, such as, not exhaustively, selected from the group consisting of EDTA (ethylenediaminetetraacetic acid), heparins and heparinoids, citrate, dimercaptosuccinic acid, dimercaptopropane sulphonic acid, acid citrate dextrose, acetylacetone, ethylenediamine, 2-(2-aminoethylamino) ethanol, diethylenetriamine, iminodiacetate, triethylenetetramine, triaminotriethylamine, nitrilotriacetate, bis(salicylidene)ethylenediamine, ethylenediaminotriacetate, diethylenetriaminepentaacetate, triethylenetetraminehexaacetate, 1,4,7,10-tetraazacyclododecane-1,4,7,10-tetraacetate, oxalate, tartrate, citrate, dimethylglyoxime, 8-hydroxyquinoline, 2,2′-bipyridine, 1,10-phenanthroline, 1,2-bis(diphenylphosphino)ethane, hirudins and hirudin analogues, apixaban, edoxaban, rivaroxaban, Coumarins, phenprocoumon, warfarin, dicumarol, pentasaccharides, antiplatelet agents, acetylsalicylic acid (ASA), clopidogrel, ticagrelor, prasugrel, GP IIb/IIIa antagonists, tirofiban, eptifibatide, bivalirudin, rivaroxaban, Dabigatran etexilate, apixaban, PPSB (prothrombin complex), antithrombin (AT III), protein C, desmopressin, DDAVP, protamine chloride, vitamin K, argatroban, danaparoid, danaparin, lomoparan, or selected from the group oxidising agents, Mercury-based stabilisers, B-5 (mercuric chloride and sodium acetate in aqueous solution), Zenker's fixative (mercuric chloride, potassium dichromate, sodium sulphate, acetic acid, water), picric acid-based stabilisers, such as Bouin's solution: Picric acid, acetic acid, formaldehyde in aqueous solution, ammonium sulphate in aqueous solution, CARNOY fixing solution (chloroform, if necessary. with iron (III) chloride or formaldehyde-alcohol-acetic acid), chromic acid, chromacetic acid, zinc salt solution.

Such stabilisers according to the invention primarily prevent blood coagulation and/or haemolysis and/or cell lysis of the blood cells.

In a further preferred embodiment, the invention relates to such blood plasma or blood serum, which is not provided from humans or animals, but which largely corresponds to a composition of blood components, the individual blood components being added in each case.

A reference material according to the invention can therefore have the following composition for the blood serum or blood plasma, as shown in Table 1, wherein in addition to serum or plasma proteins, in particular albumin, one or more blood components are added, in particular up to 5 components or 10 components or 20 components or 30 components from Table 1. In particular, in addition to albumin, up to 76 components from Table 1 can be added. The concentrations of the selected components can vary in each case, or can be selected from

Table 1 in each case.

TABLE 1
Reference values for relevant blood components in plasma/serum
according to Hahn, Checklist for Internal Medicine, 2006

| | | Preferred | |
|---|---|---|---|
| | Substance name/blood component | concentrations | |
| 1. | ACTH | 2-11 | pmol/l |
| 2. | Albumin | 35-55 | g/l |
| 3. | Alpha-amylase | <100 | U/l |
| 4. | Alpha1-fetoprotein (AFP) | <10 | ng/ml |
| 5. | Alkaline phosphatase (AP) | 65-220 | U/l |
| 6. | Ammonia | m: 11-55 | μmol/l |
| 7. | Antistreptolysin titre | <200 | IU/ml |
| 8. | Antithrombin (AT III) | 75-120% | |
| 9. | Total bilirubin | 3.4-18.8 | μmol/l |
| 10. | Bilirubin direct | 0.9-5.1 | μmol/l |
| 11. | Bilirubin indirect | <13.7 | μmol/l |
| 12. | Calcium | 2.3-2.6 | mmol/l |
| 13. | Carcino-embryonic antigen (CEA) | <3 | mg/l |
| 14. | Chloride | 98-112 | mmol/l |
| 15. | Total cholesterol | 3.1-6.5 | mmol/l |
| 16. | HDL | >1.0 | mmol/l |
| 17. | LDL | <4.0 | mmol/l |
| 18. | Cholinesterase (CHE) | 3000-8000 | U/l |
| 19. | C3 complement | 0.55-1.2 | g/l |
| 20. | C4 complement | 0.2-0.5 | g/l |
| 21. | Coeruloplasmin | 0.95-3.7 | mmol/l |
| 22. | C-peptide | 1.1-3.6 | mg/l |
| 23. | C-reactive protein (CRP) | <5 | mg/l |
| 24. | Creatine kinase (CK) | <174 | U/l |
| 25. | Creatine kinase isoenzyme MB (CK-MB) | <6% of the CK | |
| 26. | Digoxin | 0.8-2.0 | mg/l |
| 27. | Digitoxin | 15-25 | mg/l |
| 28. | Iron | 11-27 | μmol/l |
| 29. | alpha1-globulin | 1-4 | g/l |
| 30. | alpha2-globulin | 5-9 | g/l |
| 31. | beta-globulin | 6-11 | g/l |
| 32. | gamma globulin | 8-15 | g/l |
| 33. | Ferritin | 30-200 | μg/l |
| 34. | Fibrinogen | 5.9-11.8 | μmol/l |
| 35. | Folic acid | 3-15 | ng/ml |
| 36. | Total protein | 60-84 | g/l |
| 37. | Glucose fasting | 3.9-5.6 | mmol/l |
| 38. | GGT | 4-28 | U/l |
| 39. | GOT | <18 | U/l |
| 40. | GPT | <22 | U/l |
| 41. | Haptoglobin | 0.2-2.04 | g/l |
| 42. | Uric acid | 155-384 | μmol/l |
| 43. | Urea | 1.7-9.3 | mmol/l |
| 44. | alpha-HBDH | 72-182 | U/l |
| 45. | Immunoglobulin G | 8-18 | g/l |
| 46. | Immunoglobulin A | 0.9-4.5 | g/l |
| 47. | Immunoglobulin M | 0.6-2.6 | g/l |
| 48. | Potassium | 3.5-5 | mmol/l |
| 49. | Calcium | 2.3-2.6 | mmol/l |
| 50. | Creatinine | 44-106 | μmol/l |
| 51. | Copper | 11-24 | μmol/l |
| 52. | Lactate | 0.63-2.44 | mmol/l |
| 53. | LDH | 135-225 | U/l |
| 54. | LAP | 16-32 | U/l |
| 55. | Lipase | 30-180 | U/l |
| 56. | Lipoprotein (a) | <300 | mg/l |
| 57. | Magnesium | 0.7-1.6 | mmol/l |
| 58. | Sodium | 135-150 | mmol/l |
| 59. | Phosphate | 0.77-1.55 | mmol/l |
| 60. | Prostate specific antigen | <3 | μg/l |
| 61. | Rheumatoid factor | <20 | IU/ml |
| 62. | Theophylline | 10-20 | mg/l |
| 63. | TSH basal | 0.3-3.5 | mU/l |
| 64. | free thyroxine (FT4) | 7-30 | pmol/l |
| 65. | free triiodothyronine (FT3) | 4.6-9.2 | pmol/l |
| 66. | TBG | 12-30 | ug/ml |
| 67 | Thyroglobulin | <50 | ng/ml |
| 68. | Transferrin | 2.0-4.0 | g/l |
| 69. | Triglycerides | 0.83-2.3 | mmol/l |
| 70 | Vitamin A | 0.7-2.8 | μmol/l |
| 71. | Vitamin B12 | 229-812 | pmol/l |
| 72. | Vitamin D | 700-3100 | U/l |
| 73. | Vitamin E | 12-48 | μmol/l |
| 74. | Fragmented DNA, cfDNA | 1-17 ng DNA/ml | |
| 75. | Genomic DNA, gDNA | <2 | μg/ml |
| 76. | Extracellular vesicles | 2 * 10¹⁰/mL | |
| 77. | RNA | 1-1000 | ng/ml |

All in an aqueous solution. All the proteins mentioned are also called serum or plasma proteins.

In a preferred embodiment, the composition is free of antibodies, in particular free of immunoglobulins and/or free of DNA, RNA or extracellular vesicles.

Such a blood sample provided is hereinafter referred to as reference material according to the invention.

In a further preferred embodiment, the added blood plasma or blood serum has defined levels of at least one ingredient selected from the group consisting of gDNA, fragmented DNA, tumour-associated and non-tumour-associated, methylated or non-methylated cfDNA, histone-bound DNA, circular DNA, mitochondrial DNA, single-stranded or double-stranded DNA, ctDNA, RNA, methylated or non-methylated RNA, miRMA, tRNA, SCRNA, rRNA, mRNA, tumour nucleic acid, spiked nucleic acid, exosome, proteins, peptides, extracellular vesicles, exosomes, in particular with the following ranges 1-1500 ng DNA/ml, in particular with a proportion of cfDNA in pregnant women of 2-15% of foetal cfDNA, 1-1000 ng RNA/ml.

Furthermore, the added blood plasma or blood serum has defined values of at least one biological material selected from the group consisting of cells, eukaryotic cells, viruses, fungi, fungal spores, prions, bacteria, parasites, tumour cells, circulating tumour cells (CTC), circulating endothelial cells (CTEC), in particular with the following ranges 1-100.000 viruses/ml, 1-100,000 bacteria/ml, 1-100,000 fungi/ml, 1-100,000 fungal spores/ml, 1-100,000 parasites/ml, 1-1,000 tumour cells/ml or 1-1,000 endothelial cells/ml.

The aforementioned embodiments of the reference material according to the invention advantageously allow the simulation of blood samples from, for example, sick patients and other conditions.

Furthermore, it is preferred that fragmented (cell-free) DNA (cfDNA), which can occur in various sizes, is preferably used in the size ranges 140-180 base pairs (bp), 310-350 bp, 480-520 bp. The inventors were able to establish that the size and size distribution of such cfDNA in a reference material is critical, particularly for nucleic acid sequencing methods.

In a further embodiment of the invention, large cfDNA fragments preferably in the range of 2,000 to 15,000 bp can be used, while mitochondrial DNA between 40-300 bp is used. Extrachromosomal circular DNA has a size between 30-20,000 bp.

Furthermore, it is preferred that RNA, in particular miRNA, which can occur in different sizes, is preferably used in the size ranges 10-250 bp, in particular 10-500 bp. The inventors were able to establish that the size and size distribution of such RNA, in particular miRNA, in a reference material is critical, especially for nucleic acid sequencing methods.

“Blood” in the sense of the present invention can be whole blood, which comprises blood cells and blood serum. In particular, the blood can be taken from a human or an animal.

“Blood cells” in the sense of the present invention are those which remain in the residue after separation of the blood serum, for example after centrifugation, and comprise erythrocytes, leucocytes, thrombocytes and others.

In a preferred embodiment of the invention, the proportion of blood cells is 15-70% by weight, preferably 37-54% by weight of the reference material according to the invention.

A further preferred feature of the reference material according to the invention is that 99% by weight are erythrocytes (4.5−5.9×10\hat{ }6 cells/μl blood, with the lowest value applying to women and the highest value applying to men.

Another preferred feature of the reference material according to the invention is that <1% leucocytes (approx. 4,000-10,000 cells/μl blood for adults and for children: up to 17,000 cells/μl blood, infants up to 30,000 cells/μl blood) and <1% thrombocytes (approx. 150,000-400,000 cells/μl blood).

“Blood plasma” within the meaning of the present invention can be obtained by centrifuging blood and separating the supernatant, whereby the blood has previously been mixed with an anticoagulant, such as sodium citrate or EDTA. The blood plasma usually contains all coagulation and fibrinolysis factors in active form, as well as the cfDNA, which is further analysed in particular when using the liquid biopsy tests.

“Blood serum” within the meaning of the present invention is obtained by centrifuging blood and separating the supernatant. In contrast to blood plasma, it does not contain any anticoagulants.

For the purposes of the present invention, blood serum or blood plasma can also be used, in particular those which essentially comprise the following constituents:

TABLE 2
Example of a composition and concentrations of the ingredients of
a serum or plasma according to the invention (solvent is water)

| Substance name/blood | | |
|---|---|---|
| component | Concentration | |
| Plasma proteins, in particular | 50.3 | g/l |
| albumin | | |
| Electrolytes (Na)⁺ | 89.6 | mmol/l |
| Electrolytes (K)⁺ | 2.0 | mmol/l |
| Electrolytes (Ca)⁺ | 0.025 | mmol/l |
| Electrolytes (Cl)⁻ | 105.8 | mmol/l |
| Electrolytes (PO)₄³⁻ | 6.7 | mmol/l |

TABLE 3
Example of a composition and concentrations of the ingredients
of a serum or plasma according to the invention with
the addition of stabilisers (solvent is water)

| Substance name | Concentration | |
|---|---|---|
| Plasma proteins, in particular | 50.3 | g/l |
| albumin | | |
| Electrolytes (Na)+ | 89.6 | mmol/l |
| Electrolytes (K)+ | 2.0 | mmol/l |
| Electrolytes (Ca)+ | 0.025 | mmol/l |
| Electrolytes (Cl)⁻ | 105.8 | mmol/l |
| Electrolytes (PO)₄³⁻ | 6.7 | mmol/l |
| Organic acids (citrate) | 0.008 | mmol/l |
| EDTA | 9.7183 | mmol/l |

TABLE 4
Example of a composition and concentrations of the ingredients
of a serum or plasma according to the invention with
addition of stabilisers and added ingredient corresponding
to an analyte (solvent is water)

| Substance name | Concentration | |
|---|---|---|
| Plasma proteins (e.g. albumin) | 50.3 | g/l |
| Electrolytes (Na)+ | 89.6 | mmol/l |
| Electrolytes (K)+ | 2.0 | mmol/l |
| Electrolytes (Ca)+ | 0.025 | mmol/l |
| Electrolytes (Cl)⁻ | 105.8 | mmol/l |
| Electrolytes (PO)₄³⁻ | 6.7 | mmol/l |
| Organic acids (citrate) | 0.008 | mmol/l |
| EDTA | 9.7183 | mmol/l |
| Fragmented DNA | 80 ng DNA/ml | |
| Extracellular vesicles | 2 * 10¹⁰/mL | |
| RNA | 1000 | ng/ml |
| Viruses | 1000/ml | |
| Fungal spores | 1000/ml | |
| Bacteria | 1000/ml | |
| Genomic DNA | | |

TABLE 5
Example of a composition and concentrations of the
ingredients of a blood according to the invention

| Blood | Share (%) |
|---|---|
| Blood plasma | 55 |
| Blood cells | 45 |

| Blood plasma | Share (v/v) |
|---|---|
| Water | 90% |
| Plasma proteins in | 8% |
| solution | |
| thereof albumin | 60% |
| thereof globulin | 40% |
| Ions, glucose, vitamins, | |
| hormones, | |
| enzymes, urea, uric | 2% |
| acid, creatinine | |

| Blood cells | Proportion (m/m) |
|---|---|
| Erythrocytes 4-5 | 99% |
| million/μl blood | |
| Leukocytes (5-10 | <1% |
| thousand/μl blood) | |
| Thrombocytes 150- | <1% |
| 300 thousand/μl blood | |

In a further embodiment, therefore, one or more chemical substances may be added to the reference material according to the invention. Such chemical substances may be non-exhaustive, in particular at least one organic molecule which, in addition to carbon (C) and hydrogen (H), may contain heteroatoms, such as oxygen (O), nitrogen (N), sulphur(S) or phosphorus (P). The chemical substances can have linear and/or ring-shaped carbon chains including heteroatoms. Organic molecules with less than 1,000 g/mol are preferred. Such a chemical substance can be a biomarker, analyte, active ingredient, antibody, antigen or drug. In particular, an ingredient selected from the group consisting of gDNA, fragmented DNA, tumour-associated and non-tumour-associated, methylated or non-methylated cfDNA, histone-bound DNA, circular DNA, mitochondrial DNA can be added, single-stranded or double-stranded DNA, ctDNA, RNA, methylated or non-methylated RNA, miRMA, ERNA, SCRNA, rRNA, mRNA, tumour nucleic acid, spiked nucleic acid, exosome, proteins, peptides, extracellular vesicles, exosomes.

In a further embodiment, one or more biological materials may be added to the reference material according to the invention, such as cells, eukaryotic cells, viruses, fungi, fungal spores, prions, bacteria, parasites, tumour cells, circulating tumour cells (CTC), circulating endothelial cells (CTEC), which may be viable or functional.

For the purposes of the present invention, “tumour nucleic acid” means any known human wild-type sequence. Such tumour nucleic acids can be stored in databases such as
COSMIC: https://cancer.sanger.ac.uk/cosmic,
Targeted Cancer Care:
http://targetedcancercare.massgeneral.org/My-Trial-Guide/Diseases/Lung-Cancer/KRAS/G12C-(c-34G-T).aspx, or
OncoKB: https://oncokb.org/, My Cancer Genome:
https://www.mycancergenome.org/, National Centre for Biotechnology Information (NCBI)
https://www.ncbi.nlm.nih.gov/gene/.

In a preferred embodiment, the content of a tumour nucleic acid is preferably 5-1000 ng, in particular 400 ng DNA in serum or plasma, such as 400 ng/5 ml, corresponds to 80 ng/ml, corresponds to 0.08 ng/μl DNA in serum or plasma.

Furthermore, it is preferred that the tumour nucleic acid has a size of 30 bp to 500 bp.

For the purposes of the present invention, “spiked nucleic acid” means a sequence which has one or more mutations compared with a human wild-type sequence of a nucleic acid. Known mutation sequences can be used or artificial mutations can be introduced with respect to the human wild-type sequence. Furthermore, it is preferred that the spiked nucleic acid has at least one tumour marker, e.g. oncogene, with known mutations, in particular an oncogene selected from the group ABI1, ABL1, ABL2, ACKR3, ACSL3, ACVR1, ACVR2A, AFDN, AFF1, AFF3, AFF4, AK T1, AKT2, ALK, AMER1, APC, APOBEC3B, AR, ARHGAP26, ARHGEF12, ARID1A, ARI D1B, ARID2, ARNT, ASPSCR1, ASXL1, ATF1, ATIC, ATM, ATP1A1, ATP2B3, ATR, A TRX, AXIN1, AXIN2, B2M, BAP1, BARD1, BAX, BCL10, BCL11A, BCL11B, BCL2, BC L3, BCL6, BCL7A, BCL9, BCL9L, BCOR, BCORL1, BCR, BIRC3, BLM, BMPR1A, BRAF, BRCA1, BRCA2, BRD3, BRD4, BRIP1, BTG1, BTK, BUB1B, CACNA1D, CALR, CAMTA 1, CANT1, CARD11, CARS, CASP8, CBFA2T3, CBFB, CBL, CBLB, CBLC, CCDC6, CCN B1IP1, CCND1, CCND2, CCND3, CCNE1, CD274, CD74, CD79A, CD79B, CDC73, CDH 1, CDH11, CDK12, CDK4, CDK6, CDKN1B, CDKN2A, CDKN2C, CDX2, CEBPA, CHCHD7, CHD4, CHEK2, CIC, CIITA, CLIP1, CLTC, CLTCL1, CNBP, CNOT3, CNTRL, COL1A 1, COL2A1, CREB1, CREB3L1, CREB3L2, CREBBP, CRLF2, CRTC1, CRTC3, CSF3R, CTCF, CTNNB1, CUX1, CXCR4, CYLD, DAXX, DCTN1, DDB2, DDIT3, DDR2, DDX10, D DX3X, DDX5, DDX6, DEK, DICER1, DNAJB1, DNM2, DNMT3A, DROSHA, EBF1, EGFR, EIF3E, EIF4A2, ELF4, ELK4, ELL, EML4, EP300, EPAS1, EPS15, ERBB2, ERBB3, ERBB4, ERC1, ERCC2, ERCC3, ERCC4, ERCC5, ERG, ESR1, ETNK1, ETV1, ETV4, ET V5, ETV6, EWSR1, EXT1, EXT2, EZH2, EZR, FANCA, FANCC, FANCD2, FANCE, FANC F, FANCG, FAS, FAT1, FAT4, FBXO11, FBXW7, FCGR2B, FCRL4, FES, FEV, FGFR1, FGFR1OP, FGFR2, FGFR3, FGFR4, FH, FHIT, FIP1L1, FLCN, FLI1, FLT3, FLT4, F OXA1, FOXL2, FOXO1, FOXO3, FOXO4, FOXP1, FSTL3, FUBP1, FUS, GAS7, GATA1, GATA2, GATA3, GNA11, GNAQ, GNAS, GOLGA5, GOPC, GPC3, GPHN, GRIN2A, H3F3A, H3F3B, HERPUD1, HEY1, HIF1A, HIP1, HIST1H3B, HIST1H4I, HLA-A, HLF, HMGA1, HMGA2, HNF1A, HNRNPA2B1, HOOK3, HOXA11, HOXA13, HOXA9, HO XC11, HOXC13, HOXD11, HOXD13, HRAS, HSP90AA1, HSP90AB1, IDH1, IDH2, IGH, IGK, IGL, IKBKB, IKZF1, IL2, IL21R, IL6ST, IL7R, IRF4, IRS4, ITK, JAK1, J AK2, JAK3, JUN, KAT6A, KAT6B, KCNJ5, KDM5A, KDM5C, KDM6A, KDR, KDSR, KEAP 1, KIF5B, KIT, KLF4, KLF6, KLK2, KMT2A, KMT2C, KMT2D, KNL1, KRAS, KTN1, LA SP1, LATS1, LATS2, LCK, LEF1, LIFR, LMNA, LMO1, LMO2, LPP, LRIG3, LRP1B, L YL1, LZTR1, MAF, MAFB, MALT1, MAML2, MAP2K1, MAP2K2, MAP2K4, MAP3K1, MAP 3K13, MAPK1, MAX, MDM2, MDM4, MECOM, MED12, MEN1, MET, MITF, MLF1, MLH1, M LLT1, MLLT10, MLLT11, MLLT3, MLLT6, MN1, MPL, MRTFA, MSH2, MSH6, MSI2, MS N, MTCP1, MTOR, MUC1, MUTYH, MYB, MYC, MYCL, MYCN, MYD88, MYH11, MYH9, MYO 5A, MYOD1, NAB2, NBN, NCOA1, NCOA2, NCOA4, NCOR1, NCOR2, NDRG1, NF1, NF2, NFATC2, NFE2L2, NFIB, NFKB2, NFKBIE, NIN, NKX2-1,

NONO, NOTCH1, NOTCH2, NPM1, NR4A3, NRAS, NRG1, NSD1, NSD2, NSD3, NT5C2, N TRK1, NTRK3, NUMA1, NUP214, NUP98, NUTM1, NUTM2B, NUTM2D, OLIG2, P2RY8, PAFAH1B2, PALB2, PATZ1, PAX3, PAX5, PAX7, PAX8, PBRM1, PBX1, PCM1, PDCD1 LG2, PDE4DIP, PDGFB, PDGFRA, PDGFRB, PER1, PHF6, PHOX2B, PICALM, PIK3CA, PIK3CB, PIK3R1, PIM1, PLAG1, PLCG1, PML, PMS2, POLD1, POLE, POLQ, POT1, POU2AF1, POU5F1, PPARG, PPFIBP1, PPM1D, PPP2R1A, PPP6C, PRCC, PRDM1, PR DM16, PREX2, PRF1, PRKACA, PRKAR1A, PRRX1, PSIP1, PTCH1, PTEN, PTK6, PTP N11, PTPN13, PTPRB, PTPRC, PTPRK, PTPRT, QKI, RABEP1, RAC1, RAD21, RAD51 B, RAF1, RANBP2, RAP1GDS1, RARA, RB1, RBM10, RBM15, RECQL4, REL, RET, RHO A, RHOH, RMI2, RNF213, RNF43, ROS1, RPL10, RPL22, RPL5, RPN1, RSPO2, RSPO 3, RUNX1, RUNX1T1, SALL4, SBDS, SDC4, SDHA, SDHAF2, SDHB, SDHC, SDHD, SET, SETBP1, SETD2, SF3B1, SFPQ, SFRP4, SH2B3, SH3GL1, SIX1, SLC34A2, SLC45 A3, SMAD2, SMAD3, SMAD4, SMARCA4, SMARCB1, SMARCD1, SMARCE1, SMO, SND1, SOCS1, SOX2, SPEN, SPOP, SRC, SRSF2, SRSF3, SS18, SS18L1, SSX1, SSX2, SSX 4, STAG2, STAT3, STAT5B, STAT6, STIL, STK11, STRN, SUFU, SUZ12, SYK, TAF1 5, TAL1, TAL2, TBL1XR1, TBX3, TCEA1, TCF12, TCF3, TCF7L2, TCL1A, TENT5C, TERT, TET1, TET2, TFE3, TFEB, TFG, TGFBR2, TLX1, TLX3, TMEM127, TMPRSS2, TNFAIP3, TNFRSF14, TNFRSF17, TOP1, TP53, TP63, TPM3, TPM4, TPR, TRA, TRA F7, TRB, TRD, TRIM24, TRIM27, TRIM33, TRIP11, TRRAP, TSC1, TSC2, TSHR, U2 AF1, UBR5, USP6, USP8, VHL, WAS, WDCP, WIF1, WRN, WT1, WWTR1, XPA, XPC, XPO 1, YWHAE, ZBTB16, ZFHX3, ZMYM2, ZNF331, ZNF384, ZNF521, ZRSR2, A1CF, ACS L6, AKAP9, AKT3, ALDH2, ANK1, ARAF, ARHGAP5, ARHGEF10, ARHGEF10L, ASXL2, BAZ1A, BCL2L12, BCLAF1, BIRC6, BMP5, C15orf65, CASP3, CASP9, CCNC, CCR 4,CCR7, CD209, CD28, CDH10, CDH17, CDKNIA, CEP89, CHD2, CHIC2, CHST11, C LP1, CNBD1, CNTNAP2, COL3A1, COX6C, CPEB3, CRNKL1, CSF1R, CSMD3, CTNNA2, CTNND1, CTNND2, CUL3, CYP2C8, CYSLTR2, DCAF12L2, DCC, DGCR8, DUX4L1, E CT2L, EED, EIF1AX, ELF3, ELN, EPHA3, EPHA7, FAM131B, FAM135B, FAM47C, FA T3, FBLN2, FEN1, FKBP9, FLNA, FNBP1, FOXR1, GLI1, GMPS, GPC5, GRM3, HMGN2 P46, ID3, IGF2BP2, ISX, ITGAV, JAZF1, KAT7, KIAA1549, KNSTRN, LARP4B, LC P1, LEPROTL1, LHFPL6, LSM14A, MACC1, MALAT1, MB21D2, MDS2, MGMT, MNX1, M UC16, MUC4, N4BP2, NACA, NBEA, NCKIPSD, NTHL1, OMD, PABPC1, PCBP1, PMS1, POLG, PRDM2, PRKCB, PRPF40B, PTPN6, PTPRD, PWWP2A, RAD17, RALGDS, RFWD3, RGPD3, RGS7, ROBO2, S100A7, SEPT5, SEPT6, SEPT9, SETD1B, SETDB1, SGK1, SHTN1, SIRPA, SIX2, SKI, SMC1A, SNX29, SOX21, SPECC1, SRGAP3, STAG1, TEC, TFPT, TFRC, THRAP3, TNC, USP44, VAV1, VTI1A, WNK2, ZCCHC8, ZEB1, ZMYM3, ZNF429, ZNF479, ZNRF3.

Furthermore, such tumour marker sequences or oncogenes may have other artificial mutations.

For the purposes of the present invention, “validation” means that the intended diagnostics or in-vitro diagnostics are carried out using the reference material according to the invention as a reference sample, in particular to perform a calibration taking into account the device parameters or to create a calibration curve.

In a preferred embodiment of the invention, the in-vitro diagnostic is a liquid biopsy, wherein preferably PCR from a blood sample is measured against the blood sample according to the invention or the blood sample according to the invention is used for calibration, validation, diagnostics or in-vitro diagnostics.

Surprisingly, even small amounts of sample nucleic acid, e.g. from a blood sample, can be detected qualitatively and quantitatively with sufficient specificity and sensitivity to the reference material according to the invention. In the case of a liquid biopsy, this allows a particularly advantageous early statement to be made about tumour activity, in particular the probability of metastasis. A preferred sample nucleic acid is cfDNA or ctDNA from a patient or test person.

The invention therefore also relates to the advantageous use of the reference material in a method for determining the allele frequency and/or mutation rate and/or for absolute quantification by means of copy number determination of at least one sample nucleic acid by means of a sequencing method for nucleic acids. A quantitative determination of the sample nucleic acid can be carried out particularly advantageously on the basis of the calibration or validation.

Furthermore, the invention relates to the advantageous use of the reference material in a method for pre-analytical evaluation (transport, processing of a blood sample, storage/repeated use, e.g. freezing/thawing cycles, extraction procedure), analytical evaluation (accuracy/precision, repeatability/reproducibility, analytical specificity/sensitivity, detection limit, quantification limit, blank limit, linearity, interference, robustness).

The provision of the reference material according to the invention makes it particularly advantageous to validate the detection limit of the sample nucleic acids. A particularly advantageous application therefore relates to the validation of devices for carrying out a polymerase chain reaction (PCR), in particular next generation sequencing (NGS).

In this way, it is particularly advantageous to introduce a precisely predetermined amount of DNA material or concentration into the reference material according to the invention, whereby this reference material simulates a patient blood sample and is highly suitable in particular for carrying out tumour diagnostics, pregnancy diagnostics, heriditary genetic diagnostics or infection diagnostics.

“In vitro diagnosis” within the meaning of the present invention means any diagnosis outside the human or animal body (ex vivo) with the provision of suitable assays and devices. Preferred is the statement about a disease or condition of a patient.

In the context of the present invention, “patient” is understood to mean any test subject.

The invention is explained below by means of examples and figures. However, the invention is not limited to the examples and figures, but is in principle universally applicable.

### EXAMPLE 1

The individual components from Table 1 can be mixed with blood cells to provide a suitable reference material.

### EXAMPLE 2

DNA added to the reference material can be fragmented as follows in order to be able to reproduce the biological situation of a person as closely as possible.

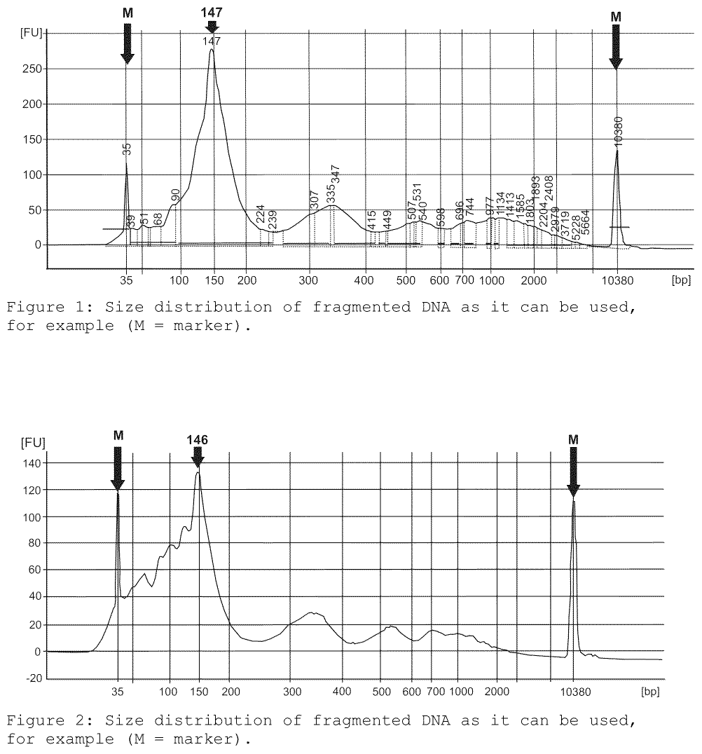

(See FIGS. 1 and 2)

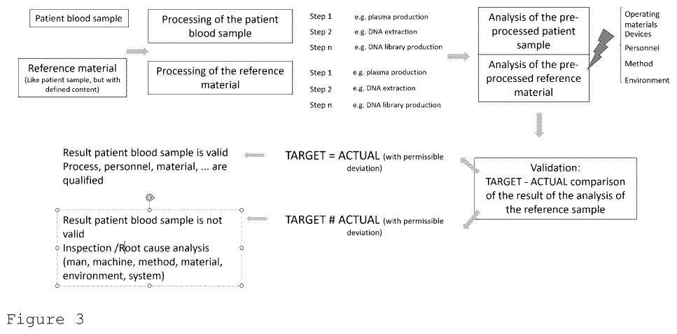

FIG. 3 shows an example of the use of the reference material in comparison to a patient blood sample.

1.-14. (canceled)

15. An in vitro diagnostic method using a reference material comprising blood in a sample comprising a sequencing method of nucleic acids, wherein a stabiliser is added, wherein the reference material simulates a patient blood sample.

16. An in vitro diagnostic method using a reference material comprising blood cells and blood plasma or blood serum in a sample comprising a sequencing method of nucleic acids, wherein a stabiliser is added, wherein the reference material simulates a patient blood sample.

17. An in vitro diagnostic method using a reference material comprising blood cells and blood plasma or blood serum comprising a sequencing method of nucleic acids, wherein the blood plasma or blood serum is composed of albumin and 5 components or up to 30 components of Table 1, wherein the reference material simulates a patient blood sample.

18. An in vitro diagnostic method using a reference comprising blood cells and blood plasma or blood serum in a sample comprising a sequencing method of nucleic acids, wherein the blood plasma or blood serum is composed of albumin and up to 76 components of Table 1, wherein the reference material simulates a patient blood sample.

19. The in vitro diagnotist method according to claim 17, wherein the blood plasma or blood serum is not provided from humans or animals.

20. A method for preparing a reference material in a sample in an in vitro diagnostic comprising a sequencing method of nucleic acids according to claim 15, wherein blood plasma or blood serum is added to blood cells, and a stabiliser is added.

21. The method according to claim 15, wherein the blood plasma or blood serum has defined values of ingredients and/or biological material.

22. The method according to claim 15, wherein the blood plasma or blood serum comprises at least one ingredient selected from the group consisting of gDNA, fragmented DNA tumour-associated and non-tumour-associated, methylated or non-methylated cfDNA, histone-bound DNA, circular DNA, mitochondrial DNA, single-stranded or double-stranded DNA, ctDNA, RNA, methylated or non-methylated RNA, miRMA, tRNA, scRNA, rRNA, mRNA, tumour nucleic acid, spiked nucleic acid, exosome, proteins, peptides, extracellular vesicles, and exosomes.

23. The method according to claim 15, wherein the blood plasma or blood serum comprises at least one biological material selected from the group consisting of cells, eukaryotic cells, viruses, fungi, fungal spores, prions, bacteria, parasites, tumour cells, circulating tumour cells (CTC), and circulating endothelial cells (CTEC).

24. The method according to claim 15, wherein the stabiliser is selected from the group consisting of crosslinking stabilisers, alcoholic stabilisers, anticoagulants, and combinations thereof.

25. The method according to claim 15, wherein at least one further chemical substance and/or at least one biological material is added.

26. The method according to claim 15, characterised in that a validation or calibration curve is created using the reference material.

27. The method according to claim 15, characterised in that the sequencing method of nucleic acids is carried out by means of PCR, qPCR (real-time quantitative polymerase chain reaction), digital droplet PCR (ddPCR) or “next generation sequencing” (NGS).

28. The method according to claim 15, characterised in that the reference material simulates a patient blood sample, in particular for carrying out tumour diagnostics, pregnancy diagnostics, heriditary genetic diagnostics or infection diagnostics.
